(19) United States
(12) Patent Application Publication
(10) Pub. No.: US 2023/0270828 A1
(43) Pub. Date: Aug. 31, 2023

(54) COMPOSITION FOR USE IN THE TREATMENT OF VIRAL INFECTIONS

(71) Applicant: BICT S.R.L., Villanova del Sillaro (LO) (IT)

(72) Inventors: Silvia Rapacioli, Villanova del Sillaro (LO) (IT); Roberto Verga, Villanova del Sillaro (LO) (IT); Simone Stasi, Villanova del Sillaro (LO) (IT); Ilaria Vitangeli, Villanova del Sillaro (LO) (IT)

(73) Assignee: BICT S.R.L., Villanova del Sillaro (LO) (IT)

(21) Appl. No.: 18/040,353

(22) PCT Filed: Jul. 30, 2021
(86) PCT No.: PCT/IB2021/056959

§ 371 Date: Feb. 2, 2023

(30) Foreign Application Priority Data
Aug. 3, 2020 (IT) 102020000019030

(51) Int. Cl. A61K 38/40; A61K 31/7048; A61K 36/87; A61K 45/06; A23L 33/105; A61K 8/64; A61K 31/785

(52) U.S. Cl. CPC A61K 38/40; A61K 31/7048; A61K 36/87; A61K 45/06; A23L 33/105; A61K 8/64; A61K 31/785

(57) ABSTRACT

A composition is described for the treatment of infections caused by pathogens, such as viruses, bacteria, fungi or yeasts. In particular, the composition of the invention comprises a synergistic association of i) active ingredient comprising lactoferrin or at least one peptide comprising lactoferricin, polylysin, natamycin or their mixture, or a mixture of lactoferrin and said at least one peptide, and ii) at least one plant extract comprising antioxidants selected from catechins, polyphenols and their mixtures. This synergistic association has been shown to be significantly active against these pathogens.

### FIELD OF THE INVENTION

The present invention relates to a composition for the treatment of infections caused by pathogens, such as viruses. In particular, the composition of the invention comprises a synergistic association of two active ingredients, which has been shown to be significantly active against such pathogens.

### BACKGROUND ART

Antibiotic resistance is a phenomenon now recognized and widespread all over the world. The development of drug resistance is a normal evolutionary process. Typically, in a colony of microbes sensitive to a certain drug, there are some that are naturally resistant: the phenomenon is called primary insensitivity. When the antibiotic destroys sensitive bacteria, those that are insensitive to the drug and which until then were in a “dormant” state begin to multiply. Or it may happen that resistance develops as a result of mutations in the genetic material of the bacterium, or the exchange of genes that confer resistance between bacteria.

Although a natural phenomenon, it is accelerated and aggravated by the misuse of antibiotic drugs. Among the practices considered most harmful is the habit of using antibiotics also to treat viral infections, where they have no use. Because viruses are tiny and replicate within cells using the cells' own mechanisms, there are only a limited number of metabolic functions that antiviral drugs can affect. Therefore, antiviral drugs are much more difficult to develop than antibiotics. Additionally, viruses can develop resistance to antiviral drugs. The same antiviral drugs can also be toxic to animals and humans. Antibiotics are not effective against viral infections, but if a person has a bacterial infection in addition to a viral infection, an antibiotic is usually needed.

Respiratory tract infections are common infections of the upper respiratory tract (e.g. nose, ears, sinuses, and throat) and lower respiratory tract (e.g. trachea, bronchi and lungs). Symptoms of upper respiratory tract infection include runny or stuffy nose, irritability, restlessness, poor appetite, decreased activity level, cough and fever.

Current therapies for respiratory tract infections involve the administration of antiviral, antibacterial, and antifungal agents, respectively, for the treatment, prevention or amelioration of viral, bacterial and fungal infections of the respiratory tract. Unfortunately, for some infections, there are no treatments available, infections have been shown to be refractory to treatment, or the occurrence of side effects outweighs the benefits of administering a therapy to a person.

It is therefore the aim of the present invention to provide an alternative solution to the use of antibiotic drugs, which allows to effectively treat infections caused by viruses, without triggering resistance phenomena.

### SUMMARY OF THE INVENTION

Said object has been achieved by a composition comprising an active ingredient and at least one plant extract, as reported in claim 1, for use in the treatment of infections caused by pathogens.

In another aspect, the present invention relates to a food supplement comprising this composition.

In another aspect, the present invention relates to a cosmetic or a medical device comprising this composition.

The characteristics and advantages of the present invention will become apparent from the following detailed description and the embodiments provided by way of illustrative and non-limiting examples.

### DETAILED DESCRIPTION OF THE INVENTION

The invention therefore relates to a composition comprising:
i) an active ingredient comprising lactoferrin or at least one peptide comprising lactoferricin, polylysin, natamycin or a mixture thereof, or a mixture of lactoferrin and said at least one peptide, and
ii) at least one plant extract comprising antioxidants selected from catechins, polyphenols and mixtures thereof,
   said composition being for use in the treatment of viral infections.

The term “treatment” refers to the effects of the composition of the invention which is capable of imparting a benefit to patients, both human and animal, suffering from an infectious disease, for example an improvement in the patient's condition or a delay in progression of the disease. This composition can also have a preventive, as well as curative effect, against infection. In this document, the term “infection” or its synonym “infectious pathology” means the invasion, colonization and/or multiplication of a microorganism within or on another host organism. The term “infection” refers to an infectious disease caused by a virus, in particular respiratory viruses, intestinal viruses and viruses that cause chronic infections and predispose to the onset of tumors.

A virus, for the purposes of the present invention, is preferably selected from respiratory syncytial virus, influenza virus, parainfluenza virus, Metapneumovirus, Rhinovirus, Adenovirus, Coronavirus, Norovirus, Rotavirus, Astrovirus, Poliovirus, Orthopoxvirus, Herpes virus, Papillomavirus, Human T virus Lymphotropic 1, Epstein-Barr Virus, Hepatitis B Virus, Hepatitis C Virus, Feline Calicivirus, or Canine Parvovirus.

Lactoferrin, also known as lactotransferrin is a multifunctional globular protein with antimicrobial activity, both bactericidal and fungicidal. Lactoferrin belongs to the transferrin family and has a molecular mass of 80 KDa, with two binding sites for the ferric ion (Fe³⁺), similar to the transferrin itself. Lactoferrin is never saturated with iron and its ferric content varies. It is found mainly in milk, but is present in many mucous secretions such as tears and saliva, and also protects infants from gastrointestinal infections. The antimicrobial activity of lactoferrin is related to its affinity for Fe³⁺(hence its high ability to compete in the free state with iron-dependent microorganisms), and to a direct action on the outer membrane of Gram negative bacteria. The combination of lactoferrin with ferric ion in mucous secretions modulates the activity and aggregation capacity of bacteria and viruses towards cell membranes. This is because some bacteria require iron to be able to carry out cell replication and lactoferrin, on the contrary, removes it from the surrounding environment, preventing its proliferation. However, bacteria such as Escherichia coli possess iron chelators which allow the microorganism to obtain it even in the presence of lactoferrin. The fungicidal and bactericidal power of lactoferrin (and of transferrin and ovotransferrin) in vitro depends on the salt concentration. In normal fresh milk, it is inactive. Lactoferrin also possesses an iron-independent bactericidal activity, being able to attack and lysate the bacterial membrane, exploiting the affinity of its cationic domains towards the bacterial membrane (negatively charged), which, in combination with lysozyme, a enzyme capable of cleaving the β1-4 glycosidic bonds of the peptidoglycan, leads to the death of the bacterium by cytolysis.

Lactoferricin is a cationic peptide that can be generated by pepsin-mediated digestion of lactoferrin, or it can be produced synthetically. The complete sequence of lactoferricin corresponds to the 17-41 fragment of lactoferrin (FKCRRWQWRM KKLGAPSITCVRRAF; LFB0084). In humans, lactoferricin corresponds to the 1-47 fragment of lactoferrin but consists of two subunits, namely fragments 1-11 and 12-47, connected by a disulfide bridge.

Polylysine or ε-polylysine is a small homo-polypeptide of the essential amino acid lysine produced by bacterial fermentation. The epsilon (ε) included in the name indicates the binding site of the lysine molecules, which unlike the normal peptides linked by the group in the α position, the amino acids are linked here by the amino group in ε and the carboxyl group by means of amide bond. E-polylysine is a homo-polypeptide of approximately 25-30 L-lysine residues. In water, polylysine contains positive charged amino acid groups. Like other surfactant compounds, it has the ability to inhibit microbiotic growth. The ε-polylysine is electrostatically absorbed on the bacterial surface which causes the dismemberment of the outer membrane.

Natamycin (international common name, also known as pimaricin) is an organic compound with the brute formula C33H47NO13. It is an antifungal agent produced by the common bacteria Streptomyces natalensis and Streptomyces chattanoogensis, and used by humans as a drug and as a food additive. Chemically, it represents a macrolide polyene epoxide, and at room temperature it appears as a white/yellow powder. Used mainly in dermatology and gynecology, natamycin is effective against fungal infections, in particular Candida, Aspergillus, Cephalosporium, Fusarium and Penicillium. It can be given as a cream, as eye drops or (for oral infections) as a lozenge. It is absorbed in negligible quantities; when taken orally, it is minimally absorbed from the gastrointestinal tract, making it unsuitable for treating systemic infections.

The composition of the invention also comprises at least one plant extract comprising antioxidants selected from catechins, polyphenols and their mixtures.

Preferably, this plant extract is grape seed extract, elderberry extract, green tea extract, grapefruit seed extract or a mixture thereof. Such extracts can be prepared by aqueous or solvent extraction.

In particular, grape seed extract is a rich source of polyphenols. These important secondary metabolites play multiple essential roles in plant physiology and exhibit a wide range of bioactive properties in the human body, primarily as an antioxidant, anti-inflammatory, anticancer, cardioprotective and anti-aging. GSE is recognized as a complex blend of flavan-3-oil monomers, oligomers, and polymers. The main monomers identified are (+)-catechin, (−)-epicatechin, (−)-epicatechin gallate, (−)-epigallocatechin and (−)-epigallocatechin gallate. The flavan-3-oil content in seed grapes is influenced by several factors, mainly cultivars, irrigation, nitrogen fertilization, delayed harvesting, and storage conditions.

Furthermore, among the polyphenols, the therapeutic action is not only expressed by flavonoids, but also by anthocyanidins. The latter are antioxidant molecules present in many phytocomplexes. One of the plants that presents them in abundance is the elder, Sambuco nigra.

Green tea comes from the leaves of a plant called Camellia sinensis, the most characteristic and main polyphenol responsible for its properties is Epigallocatechin gallate (also known by the acronym EGCG), it represents about 59% of the total polyphenols present in the leaves of dry green tea. The other components are epicatechin gallate, epigallocatechin, epicatechin and catechin, present in different percentages.

Grapefruit seed extract is obtained from the seeds and membranes of the already dehydrated fruit, which have been shown, through many scientific studies, to have an effect on a wide range of harmful microorganisms, such as bacteria, fungi, viruses, yeasts, molds, and also parasites such as worms and lice, as well as protozoa such as amoebas. Grapefruit seed extract is obtained from a standardized extract that includes the seeds and the membranous part of the grapefruit (the thin layer that contains the segments of the fruit) and can be fluid or dry, and in both cases, the extract it is rich in polyphenolic components such as quercetin, hesperidin, camphorated glycoside, neoesperidin, naringin, apigenin, rutin, poncirin, etc.

Grapefruit seed extract is considered a very powerful antiviral and bactericidal substance. The effectiveness of grapefruit seed extract on bacteria, viruses, fungi and parasites has been demonstrated thanks to multiple and significant studies carried out by Italian and worldwide laboratories and institutes, it is increasingly used in the health sector.

The mechanism of action of Grapefruit Seed Extract consists in the weakening of the structure and efficiency of the microbial cell membrane which, by changing, leads to the loss of cytoplasmic elements and becomes unable to absorb nutrients from the surrounding environment, which then kills the microorganisms due to lack of nutritional intake.

Preferably, in the composition of the invention, said active ingredient and said at least one vegetable extract are in a weight ratio of at least 1:2. It has surprisingly been found that in compositions in which said active ingredient is present in a lower quantity than said at least one plant extract, a significant antiviral synergistic effect is obtained, as also demonstrated by the examples provided below.

Preferably, in the composition of the invention, said active ingredient and said at least one vegetable extract are in a weight ratio not exceeding 1:1000.

More preferably, said active ingredient and said at least one vegetable extract are in a weight ratio of 1:5 to 1:50.

In preferred embodiments, the composition of the invention comprises 0.0005 wt % to 20 wt % of said active ingredient and 0.001 wt % to 10 wt % of said at least one vegetable extract, based on the weight of the composition.

More preferably, said active ingredient is lactoferricin.

In preferred embodiments, lactoferricin is obtained by enzymatic hydrolysis of lactoferrin, preferably by using an immobilized enzyme.

Suitable enzymes belong to the class of hydrolases which catalyze the breaking of the peptide bond between two consecutive amino acids of the protein of interest, in this case lactoferrin. These enzymes have different selectivity towards the different amino acids present, therefore there is no complete degradation of the protein into the individual amino acid constituents but rather the generation of peptide fragments of various lengths based on the position of the amino acids recognized by the hydrolytic enzyme used. Since the enzyme used for hydrolysis can represent an impurity of the product, the process is preferably based on the immobilization of the enzyme itself on inert supports through covalent bonds; the immobilization allows the removal of the biocatalyst at the end of the reaction, through physical methods (e.g. filtration) and therefore allows to avoid changes in pH and temperature increase, necessary to inactivate the free enzyme, which however have a negative impact on the activity of the product.

Preferred enzymes are proteases, in particular endoproteases including preferably pepsin, clostripaine, protease type XVII, ASP-N endopeptidase, ARG-C proteinase, Glutamyl endopeptidase, proteinase, trypsin, thermolysin, subtilisin, chymotrypsin, and their mixtures.

In preferred embodiments, said enzyme is pork pepsin, clostripaine, protease type XVII, endoprotease ASP-N, endoprotease ARG-C, or a mixture thereof.

Preferably, the pH at which to carry out the enzymatic hydrolysis is not higher than 3, more preferably is about 2.

The composition of the invention can further comprise pharmaceutically acceptable excipients. The term “excipient” refers to a compound or a mixture thereof suitable for use in a formulation for the treatment of infections caused by viruses. For example, an excipient for use in a pharmaceutical formulation generally must not cause an adverse response in a subject, nor must it significantly inhibit the effectiveness of the composition.

Suitable excipients are acidifiers, acidity regulators, anti-caking agents, antioxidants, bulking agents, strength agents, gelling agents, glazing agents, modified starches, sequestrants, thickeners, sweeteners, thinners, disaggregants, glidants, dyes, binders, lubricants, stabilizers, adsorbents, preservatives, humectants, flavors, film-forming substances, emulsifiers, wetting agents, release retardants and their mixtures.

The addition of excipients can be carried out by methods known in the art. In fact, the components can, for example, be mixed as such or with one or more excipients, enclosed in soft-gel capsules or in solid form, such as tablet, mini-tablet, micro-tablet, granule, micro-granule, pellet, multi-particulate, micronized particulate, powder, or in the form of a solution, emulsion, gel, vial, drops or spray.

The composition of the invention can be administered via oral, nasal, intra-nasal, sublingual, buccal, intramuscular, intravenous, transdermal, sub-cutaneous, external topical, internal topical, rectal, or ocular route.

Preferably, the composition of the invention further comprises at least one other natural antimicrobial peptide.

Said natural antimicrobial peptide is preferably Nisin, Beta-defensin, LL-37, Temporin A, Temporin B, Temporin L, Indolicin, Melittin, Protegrin-1, Protegrin-2, Protegrin-3, Protegrin-4, Protegrin-5, Magainin 2, Rhesus Theta-Defensin RTD-1, RTD-2, RTD-3, RTD-4, RTD-5, Arenicin-1, Arenicin-2 Arenicin-3, Dermcidin, Cecropin, Andropin, Moricin, Ceratotoxin, Dermaseptin, Bombinin, preferably Maximin H1, Maximin H2, Maximin H3, Maximin H4 or Maximin H5, Esculentin, Ranalexin, Buforin II, human CAP18, Abaecin, Apidaecin, Profenin, Bactenecin, Brevinin-1, Brevinin-2, Tachyplesin, Drosomycin, or their mixture.

In preferred embodiments, said natural antimicrobial peptide is Nisin.

Indeed, it has been observed that the composition of the invention mixed with at least one other natural antimicrobial peptide shows a further synergistic effect.

In another aspect, the present invention relates to a food supplement comprising the composition for use, as described above, said supplement being for both human and animal use.

In a further aspect, the present invention relates to lactoferricin for use in the treatment of viral infections caused by respiratory syncytial virus, influenza virus, parainfluenza virus, Metapneumovirus, Rhinovirus, Adenovirus, Coronavirus, Norovirus, Rotavirus, Astrovirus, Poliovirus, Orthopoxvirus, Herpes virus, Papillomavirus, Human T-Lymphotropic Virus 1, Epstein-Barr Virus, Hepatitis B Virus, Hepatitis C Virus, Feline Calicivirus, or Canine Parvovirus.

In a further aspect, the present invention relates to grape seed extract for use in the treatment of viral infections caused by respiratory syncytial virus, influenza virus, parainfluenza virus, Metapneumovirus, Rhinovirus, Adenovirus, Coronavirus, Norovirus, Rotavirus, Astrovirus, Poliovirus, Orthopoxvirus, Herpes virus, Papillomavirus, Human T-Lymphotropic Virus 1, Epstein-Barr Virus, Hepatitis B Virus, Hepatitis C Virus, Feline Calicivirus, or Canine Parvovirus.

In a further aspect, the present invention relates to polylysine for use in the treatment of viral infections caused by respiratory syncytial virus, influenza virus, parainfluenza virus, Metapneumovirus, Rhinovirus, Adenovirus, Coronavirus, Norovirus, Rotavirus, Astrovirus, Poliovirus, Orthopoxvirus, Herpes virus, Papillomavirus, Human T-Lymphotropic Virus 1, Epstein-Barr Virus, Hepatitis B Virus, Hepatitis C Virus, Feline Calicivirus, or Canine Parvovirus.

It should be understood that all the possible combinations of the preferred aspects of the components of the composition, as indicated above, are also described, and therefore similarly preferred.

It should be also understood that all the aspects identified as preferred and advantageous for the composition and its components are to be considered similarly preferred and advantageous also for the preparation and uses of the composition itself.

Below are working examples of the present invention provided for illustrative purposes.

### EXAMPLES

### Example 1

Below are examples of enzymatic immobilization procedures through which it is possible to obtain biocatalysts to be used in the production of lactoferrin hydrolysate including lactoferricin.

a) Enzyme Immobilization #1 (Hydrochloric Acid)

Prepare an aqueous solution of HCl at a concentration of 10 mM.

Add pork pepsin powder at a concentration of 25 mg/mL and stir until completely dissolved.

Measure the pH and bring it to 2.00±0.20 by using an aqueous solution of hydrochloric acid or sodium hydroxide.

Add epoxy resin at a concentration of 250 mg/mL to the enzymatic suspension and leave to stir for 4 h.

Remove the unbound enzyme solution by at least 3 washes with an equal volume of 10 mM HCl+1M NaCl solution.

After the last wash, remove the liquid fraction from the enzyme immobilized on the resin with a vacuum pump and store the resin at 4° C.

Titrate the enzymatic activity through standard protocol (Yoshida, F. (1956), Bull.Agri.Chem.Soc Japan 20,252-256) with 2% haemoglobin (w/v) as substrate.

b) Enzyme Immobilization #2 (Phosphoric Acid)

Prepare an aqueous solution of 85% (w/w) phosphoric acid at a concentration of 1.25% (v/v).

Add pork pepsin powder at a concentration of 25 mg/mL and stir until completely dissolved.

Measure the pH and bring it to 2.00±0.20 by using an aqueous solution of phosphoric acid or potassium hydroxide.

Add the epoxy resin at a concentration of 250 mg/mL to the enzymatic suspension and leave to stir for 4 h.

Remove the unbound enzyme solution by at least 3 washes with equal volume of 1.25% (v/v) aqueous solution of phosphoric acid+1M NaCl.

After the last wash, remove the liquid fraction from the enzyme immobilized on the resin with a vacuum pump and store the resin at 4° C.

Titrate the enzymatic activity through standard protocol (Yoshida, F. (1956), Bull.Agri.Chem.Soc Japan 20,252-256) with 2% haemoglobin (w/v) as substrate.

### Example 2

Lactoferrin Hydrolysis Process by Immobilized Enzyme #1 (HCl/Glycine)

Prepare an aqueous solution of glycine at a concentration of 3 g/L.

Keep the solution stirred and slowly pour in lactoferrin powder at a concentration of 130 g/L and wait until complete dissolution.

Correct the pH of the suspension to 2.1±0.10 by using an aqueous solution of hydrochloric acid.

Add immobilized pepsin at a concentration between 32.5-65 U/mL (measured by titration on standard substrate haemoglobin 2% (w/w)).

Less than 2 hours of reaction at a temperature of 20-30° C., the immobilized enzyme is removed through calibrated sieves that allow the liquid part (hydrolysed lactoferrin) to be efficiently separated from the solid part (immobilized exhausted enzyme).

Analyse the hydrolysis profile of the product by HPLC, from which the presence of lactoferricin is detected.

### Example 3

Lactoferrin Hydrolysis Process Using Immobilized Enzyme #2 (Phosphoric Acid)

Prepare an aqueous solution of lactoferrin at a concentration of 130 g/L, keep the solution stirred and wait until complete dissolution.

Correct the pH of the suspension to 2.1±0.10 by using an 85% (w/w) phosphoric acid solution.

Add immobilized pepsin at a concentration between 65-130 U/mL (measured by titration on a standard substrate hemoglobin 2% (w/w)).

Less than 2 hours of reaction at a temperature of 20-30° C., the immobilized enzyme is removed through calibrated sieves that allow the liquid part (hydrolyzed lactoferrin) to be efficiently separated from the solid part (immobilized exhausted enzyme).

Analyze the hydrolysis profile of the product by HPLC, from which the presence of lactoferricin is detected.

### Example 4

Lactoferrin Hydrolysis Process Using Immobilized Enzyme #3 (Lactic Acid/Phosphoric Acid)

Prepare an aqueous solution of 80% (w/w) lactic acid at a concentration of 2% (v/v).

Keep the solution stirred and slowly pour in lactoferrin powder at a concentration of 130 g/L and wait until complete dissolution.

Correct the pH of the suspension to 2.1±0.10 by using an aqueous solution of 85% (w/w) phosphoric acid.

Add immobilized pepsin at a concentration between 65-130 U/mL (measured by titration on a standard substrate haemoglobin 2% (w/w)).

Less than 2 hours of reaction at a temperature of 20-30° C., the immobilized enzyme is removed through calibrated sieves that allow the liquid part (hydrolysed lactoferrin) to be efficiently separated from the solid part (immobilized exhausted enzyme).

Analyse the hydrolysis profile of the product by HPLC, from which the presence of lactoferricin is detected.

### Example 5

Lactoferrin Hydrolysis Process Using Immobilized Enzyme #4 (Lactic Acid)

Prepare an aqueous solution of lactoferrin at a concentration of 130 g/L, keep the solution stirred and wait until complete dissolution.

Add 80% (w/w) lactic acid at a final concentration of 2% (v/v).

Add immobilized pepsin at a concentration between 32.5-65 U/mL (measured by titration on standard substrate haemoglobin 2% (w/w)).

After 2 hours of reaction at a temperature of 20-30° C., the immobilized enzyme is removed through calibrated sieves that allow the liquid part (hydrolysed lactoferrin) to be efficiently separated from the solid part (immobilized exhausted enzyme).

Analyse the hydrolysis profile of the product by HPLC, from which the presence of lactoferricin is detected.

### Example 6

Study of the In Vitro Anti SARS-CoV-2 Virus Activity of Lactoferrin/Lactoferricin and Lactoferrin

The purpose of this study was to verify the antiviral activity against SARS-CoV-2, responsible for COVID-19, of lactoferrin/lactoferricin (PREP 1) and lactoferrin (PREP 2).

Cell Culture and Cytotoxicity Assay

Cell toxicity was monitored by determining the effect of compounds against Vero cells (Monkey Kidney Epithelial Cells). Cells were maintained in DMEM medium supplemented with 10% heat-inactivated fetal calf serum, 2 mM glutamine, 100 units/ml of penicillin, 100 μg/ml of streptomycin. For the cytotoxicity assay, cells were seeded into 96-well plates at concentration of 1×10⁴ cells/well. After 24 hours of incubation, the cells were treated with serial 2 fold dilutions of PREP 1(from 8.12 to 0.06 mg/ml; 8.32 mg/ml corresponds to 1:16 dilution of the stock solution) or PREP 2 (from 9.37 mg/ml to 0.07 mg/ml; 9.37 mg/ml corresponds to 1:32 dilution of the stock solution) or chloroquine (from 200 to 1.9 μM) (as control), in a final volume of 200 μl, in duplicate.

After incubation for 72 hours at 37° C. in 5% CO₂, cell viability was measured by MTT assay.

Percentage of viable cells was calculated using untreated cells as control (100% viability) using the formula:

[(sample absorbance−cell free sample blank)/mean media control absorbance]×100.

The 50% cytotoxic concentration (CC₅₀) causing 50% reduction of Vero cells viability with respect to untreated control cells was determined using Gene5 software. Morphological changes of Vero cells were also observed by light microscopy.

Isolation of SARS-CoV-2 from Nasal-Pharyngeal Swabs

SARS-CoV-2 was isolated from 500 μl of nasal-pharyngeal swab, added to Vero cells at 80% confluence; the inoculum was removed after a 3-hour incubation at 37° C. with 5% CO₂ and the cells were incubated at 37° C., 5% CO₂, for 72 hours, when cytopathic effects (CPE) was evident.

Quantification of viral copy numbers in the cell supernatant was evaluated via specific quantitative real-time RT-PCR (qRT-PCR). SARS-CoV-2 was precipitated by means of PEG, following the manufacturer's instruction, and viral titer was determined by plaque assay, using dilution factors ranging from 10¹ to 10⁹.

The virus was used at a multiplicity of infection (MOI) of 0.05 in subsequent experiments.

Vero Cells Infection and Compounds Treatment

Vero cells were seeded into 96-well plates at a density of 1.3×10⁴ cells/well and were incubated for 24 hours at 37° C., 5% CO₂. Cells were then infected at a MOI of 0.05 (1000 PFU/well) and incubated for 2 hours at 37° C., 5% CO₂. Virus inoculum was removed and infected cells were incubated with medium for 72 hours at 37° C., 5% CO₂.

To verify antiviral activity of compounds, different schemes of treatment were used, as summarized in table 1:
1. Scheme A (preincubation of the virus with the compounds): the virus (MOI 0.05) was incubated for 1 h at 37° C. in the presence of different concentrations of PREP 1 (from 4.06 to 0.51 mg/ml) and PREP 2 (from 4.68 to 0.59 mg/ml), and then added to the cell monolayer for 2 hours at 37° C., 5% CO₂. After removal of virus inoculum, cells were incubated for 72 hours at 37° C., 5% CO₂. This scheme was repeated preincubating the different concentrations of PREP 1 and PREP 2 with the cells for 1 h at 37° C., before the viral inoculum.
2. Scheme B (treatment during virus inoculum): Cells were infected at a MOI of 0.05 (1000 PFU/well) in medium or in medium containing PREP 1 (from 4.06 to 0.51 mg/ml) or PREP 2 (from 4.68 to 0.59 mg/ml) and incubated for 2 hours at 37° C., 5% CO₂. Virus inoculum was removed and infected cells were incubated with medium for 72 hours at 37° C., 5% CO₂.
3. Scheme C (treatment of infected cells): After removal of virus inoculum from the cells, cells were treated with medium (control) or medium containing PREP 1 (from 4.06 to 0.51 mg/ml), PREP 2 (from 4.68 to 0.59 mg/ml) and incubated for 72 hours at 37° C., 5% CO₂.
4. Scheme B+C (treatment during and after virus inoculum): Cells were infected at a MOI of 0.05 (1000 PFU/well) in medium or in medium containing PREP 1 (from 4.06 to 0.51 mg/ml) or PREP 2 (from 4.68 to 0.59 mg/ml) and incubated for 2 hours at 37° C., 5% CO₂. After removal of virus inoculum from the cells, cells were treated with medium (control) or medium containing PREP 1 (from 4.06 to 0.51 mg/ml), PREP 2 (from 4.68 to 0.59 mg/ml) and incubated for 72 hours at 37° C., 5% CO₂.
5. Scheme B+C+D (treatment during, after the virus inoculum and repeated): Cells were infected at a MOI of 0.05 (1000 PFU/well) in medium or in medium containing PREP 1 (from 4.06 to 0.51 mg/ml) or PREP 2 (from 4.68 to 0.59 mg/ml) and incubated for 2 hours at 37° C., 5% CO₂. After removal of virus inoculum from the cells, cells were treated with medium (control) or medium containing PREP 1 (from 4.06 to 0.51 mg/ml), PREP 2 (from 4.68 to 0.59 mg/ml) and incubated for 2 hours at 37° C., 5% CO₂. Finally, cells were treated with medium (control) or medium containing PREP 1 (from 4.06 to 0.51 mg/ml), PREP 2 (from 4.68 to 0.59 mg/ml) and incubated for 72 hours at 37° C., 5% CO₂.

TABLE 1
Summary of the different experimental schemes

| | Pretreat- | During Virus | | |
|---|---|---|---|---|
| Experimental | ment | inoculum | Post-inoculum | Post-inoculum |
| scheme | (2 h) | (2 h) | (2 h) | (72 h) |
| A* | + | − | − | − |
| B | − | + | − | − |
| C | − | − | − | + |
| B + C | − | + | − | + |
| B + C + D | − | + | + | + |

*= pretreatment on the cells was also performed
+ = drug treatment
− = no drug, only cell culture medium

In all the experiments chloroquine (from 50 to 1.9 μM) was used as control drug

Evaluation of the Antiviral Activity of PREP 1 and PREP 2

Quantification of viral copy numbers in the cell supernatant was evaluated via specific quantitative real-time RT-PCR (qRT-PCR), after heat-treatment of the surnatants at 98° C. for 5 minutes. Results were expressed as:
the difference of cycle threshold (Ct) values of the supernatant of untreated and treated infected cells (ΔCt=Ct of the supernatant of untreated −Ct of the supernatant treated infected cells); Ct is inversely correlated to the amount of the target; ΔCt=3 corresponds to an average decreasing of viral load of 1 Log
the SARS-CoV-2 load expressed as copies/mL

Results

Compound Cytotoxicity

The cytotoxicity of PREP 1, PREP 2 and chloroquine (CQ) was measured by MTT assay.

The CC₅₀ and CC₁₀ of PREP 1 and PREP 2 are reported in Table2.

TABLE 2
CC₅₀ and CC₁₀ of PREP 1, PREP 2 and CQ

| Compound | CC₅₀ | CC₁₀ |
|---|---|---|
| PREP 1 (mg/ml) | >4.06* | >4.06* |
| PREP 2 (mg/ml) | >4.68§ | >4.68§ |
| CQ (μM) | 95.3 ± 18 | 20.93 ± 4.39 |

Data are the means ± SD of three independent experiments performed in duplicate
*1:32 dilution
§1:64 dilution
NB: Using concentrations greater than 4.06 mg/ml and 4.68 mg/ml for PREP1 and PREP2, respectively, a precipitate was observed in the wells

Based on these data, the maximum nontoxic concentration of PREP 1 and PREP 2 was assumed to be 4.06 and 4.68 mg/ml, respectively. Therefore, these concentrations and three 2-fold dilutions, were selected to test the antiviral activity.

Isolation of SARS-CoV-2 from Nasal-Pharyngeal Swabs

Isolation of the virus was confirmed by specific q-RT-PCR, and the isolated strain was subsequently titrated by plaque assay. The complete nucleotide sequence of the SARS-CoV-2 isolated strain was deposited at Gen Bank, at NCBI (accession number: MT748758)

Antiviral Activity of PREP 1 and PREP 2 Against SARS-CoV-2

Table 3 summarizes the results obtained by qRT-PCR, after heating the cell surnatants at 98° C. for 5 minutes. Results are expressed as mean ΔCt (mean of at least three experiments). A slight antiviral activity was detected for PREP 1, when added at the infected cells, at high dose, after the inoculum, one or twice (mean ΔCt −3.3) (Schemes C, B+C and B+C+D). CQ, used as control according to treatment scheme C, caused the decrease of 7.9 (50 mM) and 4.3 (16 mM) Ct, corresponding to about 2.5 and 1.5 Log.

TABLE 3
Antiviral activity of PREP 1 and PREP 2
against SARS-CoV-2 expressed as mean ΔCt

| | Concentration | | | | | |
|---|---|---|---|---|---|---|
| | of compound | Mean ΔCt | | | | |
| | (mg/ml) tested | A | B | C | B + C | B + C + D |
| PREP 1 | 0.00 | 0 | 0 | 0 | 0 | 0 |
| | 0.51 | 0.18 | 0.00 | −0.36 | −0.21 | −0.19 |
| | 1.02 | 0.16 | −0.81 | −2.00 | −0.48 | −0.59 |
| | 2.03 | 0.36 | −0.03 | −1.70 | −0.50 | −1.11 |
| | 4.06 | 0.52 | 0.22 | −3.46 | −2.90 | −3.64 |
| PREP 2 | 0.59 | 0.23 | −0.04 | −0.10 | 0.04 | −0.25 |
| | 1.17 | 0.76 | −0.04 | −1.95 | −0.08 | −0.21 |
| | 2.34 | 0.42 | 0.11 | −1.88 | −0.28 | −0.47 |
| | 4.68 | 0.25 | −0.49 | −1.92 | −1.52 | −2.47 |
| Chloroquine | 50 mM | | | −7.9 | | |
| (control) | 16 mM | | | −4.3 | | |

### Example 7

Study of the In Vitro Anti SARS-CoV-2 Virus Activity of Polylysine

The purpose of this study was to verify the antiviral activity against SARS-CoV-2, responsible for COVID-19, of polylysine (PREP 3)

Cell Culture and Cytotoxicity Assay

Cell toxicity was monitored by determining the effect of the compound against Vero cells (Monkey Kidney Epithelial Cells). Cells were maintained in DMEM medium supplemented with 10% heat-inactivated foetal calf serum, 2 mM glutamine, 100 units/ml of penicillin, 100 g/ml of streptomycin.

For the cytotoxicity assay, cells were seeded into 96-well plates at concentration of 1×10⁴ cells/well. After 24 hours of incubation, the cells were treated with serial 2 fold dilutions of PREP 3 (from 0.80 to 0.025 mg/ml) or chloroquine (from 200 to 1.9 μM) (as control), in a final volume of 200 μl, in duplicate.

After incubation for 72 hours at 37° C. in 5% CO₂, cell viability was measured by MTT assay.

Percentage of viable cells was calculated using untreated cells as control (100% viability) using the formula:

[(sample absorbance−cell free sample blank)/mean media control absorbance]×100.

The 50% cytotoxic concentration (CC₅₀) causing 50% reduction of Vero cells viability with respect to untreated control cells was determined using Gene5 software. Morphological changes of Vero cells were also observed by light microscopy.

Isolation of SARS-CoV-2 from Nasal-Pharyngeal Swabs

SARS-CoV-2 was isolated from 500 μl of nasal-pharyngeal swab, added to Vero cells at 80% confluence; the inoculum was removed after a 3-hour incubation at 37° C. with 5% CO₂ and the cells were incubated at 37° C., 5% CO₂, for 72 hours, when cytopathic effects (CPE) was evident.

Quantification of viral copy numbers in the cell supernatant was evaluated via specific quantitative real-time RT-PCR (qRT-PCR). SARS-CoV-2 was precipitated by means of PEG, following the manufacturer's instruction, and viral titer was determined by plaque assay, using dilution factors ranging from 10¹ to 10⁹.

The virus was used at a multiplicity of infection (MOI) of 0.05 in subsequent experiments.

Vero Cells Infection and Compounds Treatment

Vero cells were seeded into 96-well plates at a density of 1.3×10⁴ cells/well and were incubated for 24 hours at 37° C., 5% CO₂. Cells were then infected at a MOI of 0.05 (1000 PFU/well) and incubated for 2 hours at 37° C., 5% CO₂. Virus inoculum was removed and infected cells were incubated with medium for 72 hours at 37° C., 5% CO₂.

To verify antiviral activity of compounds, different schemes of treatment were used, as summarized in table 1:
1. Scheme A (preincubation of the virus with the compounds): the virus (MOI 0.05) was incubated for 1 h at 37° C. in the presence of different concentrations of PREP 3 (from 0.40 to 0.05 mg/ml) and then added to the cell monolayer for 2 hours at 37° C., 5% CO₂. After removal of virus inoculum, cells were incubated for 72 hours at 37° C., 5% CO₂. This scheme was repeated preincubating the different concentrations of PREP 3 with the cells for 1 h at 37° C., before the viral inoculum.
2. Scheme B (treatment during virus inoculum): Cells were infected at a MOI of 0.05 (1000 PFU/well) in medium or in medium containing PREP 3 (from 0.40 to 0.05 mg/ml) and incubated for 2 hours at 37° C., 5% CO₂. Virus inoculum was removed and infected cells were incubated with medium for 72 hours at 37° C., 5% CO₂.
3. Scheme C (treatment of infected cells): After removal of virus inoculum from the cells, cells were treated with medium (control) or medium containing PREP 3 (from 0.40 to 0.05 mg/ml) and incubated for 72 hours at 37° C., 5% CO₂.
4. Scheme B+C (treatment during and after virus inoculum): Cells were infected at a MOI of 0.05 (1000 PFU/well) in medium or in medium containing PREP 3 (from 0.40 to 0.05 mg/ml) and incubated for 2 hours at 37° C., 5% CO₂. After removal of virus inoculum from the cells, cells were treated with medium (control) or medium containing PREP 3 (from 0.40 to 0.05 mg/ml), and incubated for 72 hours at 37° C., 5% CO₂.
5. Scheme B+C+D (treatment during, after the virus inoculum and repeated): Cells were infected at a MOI of 0.05 (1000 PFU/well) in medium or in medium containing PREP 3 (from 0.40 to 0.05 mg/ml), and incubated for 2 hours at 37° C., 5% CO₂. After removal of virus inoculum from the cells, cells were treated with medium (control) or medium containing PREP 3 (from 0.40 to 0.05 mg/ml), and incubated for 2 hours at 37° C., 5% CO₂. Finally, cells were treated with medium (control) or medium containing PREP 3 (from 0.40 to 0.05 mg/ml), and incubated for 72 hours at 37° C., 5% CO₂.

TABLE 1
Summary of the different experimental schemes

| | Pretreat- | During Virus | | |
|---|---|---|---|---|
| Experimental | ment | inoculum | Post-inoculum | Post-inoculum |
| scheme | (2 h) | (2 h) | (2 h) | (72 h) |
| A* | + | − | − | − |
| B | − | + | − | − |
| C | − | − | − | + |
| B + C | − | + | − | + |
| B + C + D | − | + | + | + |

*= pretreatment on the cells was also performed
+ = drug treatment
− = no drug, only cell culture medium

In all the experiments chloroquine (from 50 to 1.9 μM) was used as control drug

Evaluation of the Antiviral Activity of PREP 3

Quantification of viral copy numbers in the cell supernatant was evaluated via specific quantitative real-time RT-PCR (qRT-PCR), after heat-treatment of the surnatants at 98° C. for 5 minutes. Results were expressed as:
the difference of cycle threshold (Ct) values of the supernatant of untreated and treated infected cells (ΔCt=Ct of the supernatant of untreated −Ct of the supernatant treated infected cells); Ct is inversely correlated to the amount of the target; ΔCt=3 corresponds to an average decreasing of viral load of 1 Log;
the SARS-CoV-2 load expressed as copies/mL.

In order to verify the virucide activity of the compound, plaque assay was conducted after the virus+compound inoculum (400 l/well) on the cells, plated in a 6 well plate. Briefly, after 2 h of inoculum with virus suspension, the inoculum was removed and the cells were covered with 0.3% agarose dissolved in cell medium and incubated for 72 hours at 37° C., 5% CO₂. Cells were fixed with 4% formaldehyde solution and, after agarose removal, stained with methylene blue. Plaques were counted and results are expressed as Plaque Forming Unit (PFU)/mL, and as percentage of virus replication, compared to untreated infected cells.

Results

Compound Cytotoxicity

The cytotoxicity of PREP 3 and chloroquine (CQ) was measured by MTT assay.

The CC₅₀ and CC₁₀ of PREP 3 are reported in Table2.

TABLE 2
CC₅₀ and CC₁₀ of PREP 3 and CQ

| Compound | CC₅₀ | CC₁₀ |
|---|---|---|
| PREP 3 (mg/ml) | 0.56 ± 0.21 | >0.40 |
| CQ (μM) | 95.3 ± 18 | 20.93 ± 4.39 |

Data are the means ± SD of three independent experiments performed in duplicate

Based on these data, the maximum nontoxic concentration of PREP 3 was assumed to be 0.4 mg/ml, therefore this concentration and the three subsequent concentrations (0.20, 0.10, 0.05 mg/ml), were selected to test the antiviral activity.

Isolation of SARS-CoV-2 from Nasal-Pharyngeal Swabs

Isolation of the virus was confirmed by specific q-RT-PCR, and the isolated strain was subsequently titrated by plaque assay. The complete nucleotide sequence of the SARS-CoV-2 isolated strain was deposited at Gen Bank, at NCBI (accession number: MT748758)

Antiviral Activity of PREP 3 Against SARS-CoV-2

Table 3 summarizes the results obtained by qRT-PCR, after isolating the RNA from the surnatant of the infected cells. Results are expressed as mean ΔCt (mean of at least three experiments). While no effect was evident in the schemes A, B and C, a dose-dependent antiviral effect was present when PREP 3 was added during and after the inoculum (scheme B+C), from −1.80 to −5.22 ΔCt, with increasing dose of PREP 3. A slight effect was also shown with the addition of a third dose of PREP 3. No antiviral effect was observed when PREP 3 was used as pretreatment of uninfected cells (data not shown).

CQ, used as control, caused the decrease of 7.9 (50 mM) and 4.3 (16 mM) Ct, corresponding to about 2.5 and 1.5 Log.

TABLE 3
Antiviral activity of PREP 3 against
SARS-COV-2 expressed as mean ΔCt

| Concentration | | | | | |
|---|---|---|---|---|---|
| of compound | Mean ΔCt | | | | |
| (mg/ml) tested | A | B | C | B + C | B + C + D |
| 0.00 | 0 | 0 | 0 | 0 | 0 |
| 0.05 | −0.27 | 0.01 | 0.31 | −1.80 | −0.38 |
| 0.10 | 0.37 | 0.29 | −0.07 | −2.44 | −0.49 |
| 0.20 | 0.02 | 0.51 | −0.50 | −2.99 | −1.71 |
| 0.40 | −0.12 | 0.57 | −1.73 | −5.22 | −2.78 |
| Chloroquine | | | −7.9 | | |
| (control) 50 mM | | | | | |
| Chloroquine | | | −4.3 | | |
| (control) 16 mM | | | | | |

The decrease in the viral replication was of more than 1 log, when the PREP 3 was used following the scheme B+C, corresponding to a maximum of 93% of inhibition of the virus replication.

Virucide Activity

To confirm the virucide activity of PREP 3, plaque assay was conducted. Table 6 reports the obtained results, expressed as mean PFU/ml. SARS-CoV-2 infectivity was reduced by 96.3%, 48.8%, 30.2% and 20%, using 0.40, 0.20, 0.1 and 0.05 mg/ml of PREP 3. These results strongly confirmed the dose-dependent effect of PREP 3, both for its antiviral and virucide activities.

TABLE 4
virucide activity of PREP 3, following the scheme B + C

| | Mean PFU/ml (%) | | | |
|---|---|---|---|---|
| Untreated | 0.05 | 0.10 | 0.20 | 0.40 |
| infected cells | mg/ml | mg/ml | mg/ml | mg/ml |
| 73.75 (100%) | 59 (80%) | 51.5 (69.8%) | 37.75 (51.2%) | 2.75 (3.7%) |

Antiviral and virucide activities against SARS-CoV-2 was observed when PREP 3 was added during and after the virus inoculum, reaching more than 90% of virus inhibition.

### Example 8

Study of the In Vitro Anti SARS-CoV-2 Virus Activity of Grape Seed Extract

The purpose of this study was to verify the antiviral activity against SARS-CoV-2, responsible for COVID-19, of grape seed (PREP 4).

Cell culture and cytotoxicity assay Cell toxicity was monitored by determining the effect of PREP4 against Vero cells (Monkey Kidney Epithelial Cells). Cells were maintained in DMEM medium supplemented with 10% heat-inactivated foetal calf serum, 2 mM glutamine, 100 units/ml of penicillin, 100 μg/ml of streptomycin.

For the cytotoxicity assay, cells were seeded into 96-well plates at concentration of 1×10⁴ cells/well.

After 24 hours of incubation, the cells were treated with serial 2 fold dilutions of PREP 4 (from 0.6 to 0.015 mg/ml) or chloroquine (from 200 to 1.9 μM) (as control), in a final volume of 200 μl, in duplicate.

After incubation for 72 hours at 37° C. in 5% CO₂, cell viability was measured by MTT assay.

Percentage of viable cells was calculated using untreated cells as control (100% viability) using the formula:

[(sample absorbance−cell free sample blank)/mean media control absorbance]×100.

The 50% cytotoxic concentration (CC₅₀) causing 50% reduction of Vero cells viability with respect to untreated control cells was determined using Gene5 software. Morphological changes of Vero cells were also observed by light microscopy.

Isolation of SARS-CoV-2 from Nasal-Pharyngeal Swabs

SARS-CoV-2 was isolated from 500 μl of nasal-pharyngeal swab, added to Vero cells at 80% confluence; the inoculum was removed after a 3-hour incubation at 37° C. with 5% CO₂ and the cells were incubated at 37° C., 5% CO₂, for 72 hours, when cytopathic effects (CPE) was evident.

Quantification of viral copy numbers in the cell supernatant was evaluated via specific quantitative real-time RT-PCR (qRT-PCR). SARS-CoV-2 was precipitated by means of PEG, following the manufacturer's instruction, and viral titer was determined by plaque assay, using dilution factors ranging from 10¹ to 10⁹.

The virus was used at a multiplicity of infection (MOI) of 0.05 in subsequent experiments.

Vero Cells Infection and Compounds Treatment

Vero cells were seeded into 96-well plates at a density of 1.3×10⁴ cells/well and were incubated for 24 hours at 37° C., 5% CO₂. Cells were then infected at a MOI of 0.05 (1000 PFU/well) and incubated for 2 hours at 37° C., 5% CO₂. Virus inoculum was removed and infected cells were incubated with medium for 72 hours at 37° C., 5% CO₂.

To verify antiviral activity of compounds, different schemes of treatment were used, as summarized in table 1:
1. Scheme A (preincubation of the virus with the compounds): the virus (MOI 0.05) was incubated for 1 h at 37° C. in the presence of different concentrations of PREP 4 (from 0.30 to 0.03 mg/ml) and then added to the cell monolayer for 2 hours at 37° C., 5% CO₂. After removal of virus inoculum, cells were incubated for 72 hours at 37° C., 5% CO₂. This scheme was repeated preincubating the different concentrations of PREP 4 with the cells for 1 h at 37° C., before the viral inoculum.
2. Scheme B (treatment during virus inoculum): Cells were infected at a MOI of 0.05 (1000 PFU/well) in medium or in medium containing PREP 4 (from 0.30 to 0.03 mg/ml) and incubated for 2 hours at 37° C., 5% CO₂. Virus inoculum was removed and infected cells were incubated with medium for 72 hours at 37° C., 5% CO₂.
3. Scheme C (treatment of infected cells): After removal of virus inoculum from the cells, cells were treated with medium (control) or medium containing PREP 4 (from 0.30 to 0.03 mg/ml) and incubated for 72 hours at 37° C., 5% CO₂.
4. Scheme B+C (treatment during and after virus inoculum): Cells were infected at a MOI of 0.05 (1000 PFU/well) in medium or in medium containing PREP 4 (from 0.30 to 0.03 mg/ml) and incubated for 2 hours at 37° C., 5% CO₂. After removal of virus inoculum from the cells, cells were treated with medium (control) or medium containing PREP 4 (from 0.30 to 0.03 mg/ml), and incubated for 72 hours at 37° C., 5% CO₂.
5. Scheme B+C+D (treatment during, after the virus inoculum and repeated): Cells were infected at a MOI of 0.05 (1000 PFU/well) in medium or in medium containing PREP 4 (from 0.30 to 0.03 mg/ml), and incubated for 2 hours at 37° C., 5% CO₂. After removal of virus inoculum from the cells, cells were treated with medium (control) or medium containing PREP 4 (from 0.30 to 0.03 mg/ml), and incubated for 2 hours at 37° C., 5% CO₂.

Finally, cells were treated with medium (control) or medium containing PREP 4 (from 0.30 to 0.03 mg/ml), and incubated for 72 hours at 37° C., 5% CO₂.

TABLE 1
Summary of the different experimental schemes

| | Pretreat- | During Virus | | |
|---|---|---|---|---|
| Experimental | ment | inoculum | Post-inoculum | Post-inoculum |
| scheme | (2 h) | (2 h) | (2 h) | (72 h) |
| A* | + | − | − | − |
| B | − | + | − | − |
| C | − | − | − | + |
| B + C | − | + | − | + |
| B + C + D | − | + | + | + |

*= pretreatment on the cells was also performed
+ = drug treatment
− = no drug, only cell culture medium

In all the experiments chloroquine (from 50 to 1.9 μM) was used as control drug, following the scheme of treatment C.

Evaluation of the Antiviral and Virucide Activity of PREP 4

Quantification of viral copy numbers in the cell supernatant was evaluated via specific quantitative real-time RT-PCR (qRT-PCR), after heat-treatment of the surnatants at 98° C. for 5 minutes. Results were expressed as:
the difference of cycle threshold (Ct) values of the supernatant of untreated and treated infected cells (ΔCt=Ct of the supernatant of untreated −Ct of the supernatant treated infected cells); Ct is inversely correlated to the amount of the target; ΔCt=3 corresponds to an average decreasing of viral load of 1 Log;
the SARS-CoV-2 load expressed as copies/mL.

In order to verify the virucide activity of the compound, plaque assay was conducted after the virus+compound inoculum (400 l/well) on the cells, plated in a 6 well plate. Briefly, after 2 h of inoculum with virus suspension, the inoculum was removed and the cells were covered with 0.3% agarose dissolved in cell medium and incubated for 72 hours at 37° C., 5% CO₂. Cells were fixed with 4% formaldehyde solution and, after agarose removal, stained with methylene blue. Plaques were counted and results are expressed as Plaque Forming Unit (PFU)/mL, and as percentage of virus replication, compared to untreated infected cells.

Results

Compound Cytotoxicity

The cytotoxicity of PREP 4 and chloroquine (CQ) was measured by MTT assay.

The CC₅₀ and CC₁₀ of PREP 4 are reported in Table2.

TABLE 2
CC₅₀ and CC₁₀ of PREP 4 and CQ

| Compound | CC₅₀ | CC₁₀ |
|---|---|---|
| PREP 4 (mg/ml) | 0.175 ± 0.07 | >0.30 |
| CQ (μM) | 95.3 ± 18 | 20.93 ± 4.39 |

Data are the means ± SD of three independent experiments performed in duplicate

Based on these data, the maximum nontoxic concentration of PREP 4 was assumed to be 0.30 mg/ml, therefore this concentration and the three subsequent concentrations (0.15, 0.08, 0.04 mg/ml), were selected to test the antiviral activity.

Isolation of SARS-CoV-2 from Nasal-Pharyngeal Swabs

Isolation of the virus was confirmed by specific q-RT-PCR, and the isolated strain was subsequently titrated by plaque assay. The complete nucleotide sequence of the SARS-CoV-2 isolated strain was deposited at Gen Bank, at NCBI (accession number: MT748758)

Antiviral Activity of PREP 4 Against SARS-CoV-2

Table 3 summarizes the results obtained by qRT-PCR, after heating the cell surnatants at 98° C. for 5 minutes. Results are expressed as mean ΔCt (mean of at least three experiments). While no antiviral activity was observed when the PREP 4 was added to the cells, following the schemes A and B, a strong antiviral activity was observed when PREP 4 was added after the viral inoculum: ΔCt of −1.89, −4.23, −8.69 and −14.41 were detected in correspondence of the addition of 0.04, 0.08, 0.15 and 0.30 mg/ml of PREP 4, respectively. The same trend, but slightly inferior was seen when PREP 4 was added during the inoculum and after it, once or twice (schemes B+C and B+C+D). CQ, used as control, caused the decrease of 7.9 (50 mM) and 4.3 (16 mM) Ct, corresponding to about 2.5 and 1.5 Log.

TABLE 3
Antiviral activity of PREP 4 against
SARS-COV-2 expressed as mean ΔCt

| Concentration | | | | | |
|---|---|---|---|---|---|
| of compound | Mean ΔCt | | | | |
| (mg/ml) tested | A | B | C | B + C | B + C + D |
| 0.00 | 0 | 0 | 0 | 0 | 0 |
| 0.04 | 0.17 | −0.07 | −1.89 | −0.52 | −0.64 |
| 0.08 | 0.07 | −0.13 | −4.23 | −1.71 | −1.39 |
| 0.15 | 0.21 | 0.05 | −8.69 | −4.90 | −4.27 |
| 0.30 | 0.07 | 0.14 | −14.41 | −9.59 | −12.39 |
| Chloroquine | | | −7.9 | | |
| (control) 50 mM | | | | | |
| Chloroquine | | | 4.3 | | |
| (control) 16 mM | | | | | |

The strong antiviral activity of PREP 4 is evident, since it induced the decreased of up to 3 Log when added after the inoculum, once or twice.

This decrease corresponds to a complete (100%) antiviral activity.

Virucide Activity

To confirm the virucide activity of PREP 4, plaque assay was conducted, following the C scheme.

Table 4 reported the obtained results, expressed as mean PFU/ml. SARS-CoV-2 infectivity was reduced by 100% using 0.30 mg/ml and 0.15 mg/m, and 81.5% and 52%, using 0.08 mg/ml, and 0.04 mg/ml of PREP 4, respectively.

These results strongly confirmed the dose-dependent effect of PREP 4, both for its antiviral and virucide activities. Additionally, they resembled the antiviral activity of the CQ, used as control (data not shown).

TABLE 4
Virucide activity of PREP 4

| Untreated | Mean PFU/ml (%) | | | |
|---|---|---|---|---|
| infected cells | 0.04 mg/ml | 0.08 mg/ml | 0.15 mg/ml | 0.30 mg/ml |
| 67.5 (100%) | 32.5 (48%) | 12.5 (18.5%) | 0 (0%) | 0 (0%) |

Antiviral and virucide activities against SARS-CoV-2 was observed when PREP 4 was added after the virus inoculum, reaching 100% of virus inhibition.

### Example 9

Activities Against RNA and DNA Viruses. Enveloped and Non Enveloped. Tested According to ISO EN 14476: 2013-ISO EN 16777:2019 and ISO 18184:2019

| | | Virus | Tested cells |
|---|---|---|---|
| EN | RNA | Murine norovirus | RAW 264.7 |
| 14476: | non | S99 Berlin | ATCC TIB-71 |
| 2013 - | enveloped | Poliovirus type 1 | Hela |
| ISO EN | | LSc 2 ab (Picornavirus) | ATCC CCL-2 |
| 16777: | DNA | Adenovirus | Hela |
| 2019 | non- | ATCC VR-5 | ATCC CCL-2 |
| | enveloped | Murine Parvovirus | A9 cells |
| | | ATCC VR-1346 | ATCC CCL-1.4 |
| | DNA | Vaccinia virus Ankara (MVA) | BHK-21 |
| | enveloped | ATCC VR-1508 | ATCC CCL-10 |
| | | Vaccinia virus strain Elstree | Vero |
| | | ATCC VR-1549 | ATCC CCL-81 |
| | | Herpes virus canino | Cf2Th |
| | | CHV-1 | ATCC CRL 6574 |
| ISO | RNA | Feline calicivirus | CRFK |
| 18184: | non | F-9 ATCC VR-782 | ATCC CCL-94 |
| 2019 | enveloped | | |
| | RNA | Influenza A virus H1N1 | MDCK cell |
| | enveloped | ATCC VR-1469 | ATCC CCL-34 |
| | | Influenza A virus H3N2 | MDCK cell |
| | | ATCC-VR-1679 | ATCC CCL-34 |

### Example 9A) Lactoferricin and Grape Seed Against Herpes Virus

With the experiment reported here, the synergistic effect of the antiviral activity of lactoferricin in combination with Grape Seed extract was evaluated. This antiviral activity was evaluated using Cf2Th cells (canine thymocytes) infected with canine herpes virus, CHV-1.

Briefly, the Cf2Th cells were plated at a concentration of 5×10⁵ cells/mL in 96-well multiwells (100 μL/well) and incubated at 37° C. in an atmosphere with 5% CO₂.

Upon reaching about 90% confluence (48 h incubation), the medium was aspirated from each well, and replaced with 100 μL fresh medium containing a final virus concentration equal to 1×10³ UFP/mL, and different concentrations of Lactoferricin and Grape Seed extract.

In particular, the different combinations between the two compounds had been previously prepared by crossing serial dilutions of the same, according to the protocol defined “broth microdilution checkerboard method”.

The cells were left in co-incubation with the virus and the compounds under examination for 48 h (37° C. -5% CO₂).

At the end of the incubation, the wells characterized by a cell lysis given by the viral infection were discriminated from those in which the virus was inhibited by the compounds and therefore with viable cells. This evaluation was carried out both by direct observation under the microscope and by staining with methylene blue.

According to the staining protocol, the medium containing the various treatments was aspirated from each well, and the cells were washed with PBS, thus eliminating all non-adherent cells.

Subsequently, the cells were fixed by incubating each well with 100 μL of a 10% formaldehyde aqueous solution for 20 minutes at room temperature.

The fixative solution was then aspirated and the cells washed with PBS, before adding 100 μL of a solution of methylene blue (1% in water) to each well.

After 15 minutes of incubation at room temperature, the methylene blue solution was removed and the cells washed with PBS to remove excess dye.

In this way, in the wells that contained viable cells, these are intensely stained, while the wells in which the cells had undergone viral lysis show no staining.

| Compound | Lactoferricin | Grape Seed Extract |
|---|---|---|
| Minimum Antiviral Concentration | 3.125% | 0.063 g/L |
| Compound alone | | |
| Minimum Antiviral Concentration | 0.781% | 0.031 g/L |
| Compound in association | | |

To determine the actual presence of synergy between the two compounds, the ratio below was calculated, defined as the FIC Index (FIC I). In order for the association of the two compounds in question to be considered synergistic, the FIC Index must be less than 1.

$$FIC I = \frac{active concentration compound A in association}{active concentrationtion compound A alone} + \frac{active concentration compound B in association}{active concentration compound B alone}$$

Therefore, considering the results obtained in the present experiment, the value of the FIC Index can be determined, as reported below.

$$FIC I = \frac{0.781 \%}{3.125 \%} + \frac{0.031 g / L}{0.063 g / L} = 0.75$$

This demonstrates the presence of a synergistic effect of the antiviral activity of lactoferricin in combination with Grape Seed extract.

1. A method of treating viral infections, the method comprising the step of administering to a subject in need thereof an effective amount of a composition comprising:
i) 0.0005 wt % to 20 wt % of an active ingredient comprising lactoferrin or at least one peptide comprising lactoferricin, polylysine, natamycin or a mixture thereof, or a mixture of lactoferrin and said at least one peptide, and
ii) 0.001 wt % to 10 wt % of at least one plant extract comprising antioxidants selected from catechins, polyphenols and their mixtures, said plant extract being grape seed extract, elderberry extract, green tea extract, grapefruit seed extract or a mixture thereof.

2. The method of claim 1, wherein said active ingredient and said at least one plant extract are in a weight ratio of at least 1:2.

3. The method of claim 1, wherein said active ingredient and said at least one plant extract are in a weight ratio not higher than 1:1000.

4. The method of claim 3, wherein said active ingredient and said at least one plant extract are in a weight ratio of 1:5 to 1:50.

5. (canceled)

6. The method of claim 1, wherein said active ingredient is lactoferricin.

7. The method of claim 1, wherein lactoferricin is obtained by enzymatic hydrolysis of lactoferrin; by use of an immobilized enzyme.

8. The method of claim 1, further comprising at least one other natural antimicrobial peptide.

9. The method of claim 1, wherein said viral infections are caused by respiratory syncytial virus, influenza virus, parainfluenza virus, Metapneumovirus, Rinovirus, Adenovirus, Coronavirus, Norovirus, Rotavirus, Astrovirus, Poliovirus, Orthopoxvirus, Herpes virus, Papillomavirus, Human T-lymphotropic virus 1, Epstein-Barr virus, Hepatitis B virus, Hepatitis C virus, Feline Calicivirus, or canine Parvovirus.

10. The method of claim 8, wherein said at least one other natural antimicrobial peptide is selected from Nisin, Beta-defensin, LL-37, Temporin A, Temporin B, Temporin L, Indolicin, Melittin, Protegrin-1, Protegrin-2, Protegrin-3, Protegrin-4, Protegrin-5, Magainin 2, Rhesus Theta-Defensin RTD-1, RTD-2, RTD-3, RTD-4, RTD-5, Arenicin-1, Arenicin-2 Arenicin-3, Dermcidin, Cecropin, Andropin, Moricin, Ceratotoxin, Dermaseptin, Bombinin, preferably Maximin H1, Maximin H2, Maximin H3, Maximin H4 or Maximin H5, Esculentin, Ranalexin, Buforin II, human CAP18, Abaecin, Apidaecin, Profenin, Bactenecin, Brevinin-1, Brevinin-2, Tachyplesin, Drosomycin, or their mixture.

11. (canceled)

12. The method of claim 1, wherein said composition is in the form of a food supplement, cosmetic product or medical device.
